(19) United States
(12) Patent Application Publication
(10) Pub. No.: US 2015/0085203 A1
(43) Pub. Date: Mar. 26, 2015

(54) DOUBLE-VISION TOUCH DISPLAY DEVICE AND MANUFACTURING METHOD THEREOF

(71) Applicant: BOE TECHNOLOGY GROUP CO., LTD., Beijing (CN)

(72) Inventors: Wenbo Li, Beijing (CN); Liugang Zhou, Beijing (CN); Naifu Wu, Beijing (CN)

(21) Appl. No.: 14/342,161

(22) PCT Filed: Dec. 18, 2013
(86) PCT No.: PCT/CN2013/089893

§ 371 Date: Feb. 28, 2014

(30) Foreign Application Priority Data
May 16, 2013 (CN) 201310182419.9

(51) Int. Cl. G02F 1/13; G02F 1/1343; G06F 3/044; A61B 3/113; G02F 1/1333; G02F 1/1335

(52) U.S. Cl. CPC G02F 1/1323; G02F 1/13338; G02F 1/133512; G06F 3/044; A61B 3/113; G02F 1/133514; G02F 1/13439
USPC 349/12; 445/46

(57) ABSTRACT

Embodiments of the present invention provide a double-vision touch display device and a manufacturing method thereof, and the double-vision touch display device comprises: a touch circuit; a grating, wherein, the touch circuit comprises: first touch electrodes, second touch electrodes, and a touch control module, and the first touch electrodes cross with and are insulated from the second touch electrodes, and the first touch electrodes and the second touch electrodes are connected with the touch control module, respectively, and the grating is made from an opaque conductive material and formed with the first touch electrodes synchronously.

### TECHNICAL FIELD

Embodiments of the present invention relate to a double-vision touch display device and a manufacturing method thereof.

### BACKGROUND

Double-vision displaying refers to displaying different images by the same one display at different angles, that is, a user can see different images on one display screen at different angles, and a double-vision display can be used as a vehicle display. For example, by using the double-vision display, passengers at different seats in a vehicle can see different images from one display respectively, thus it is not necessary to provide each passenger with a separate display, and the cost can be lowered and the space within the vehicle can be saved.

As shown in FIG. 1, a structure of a double-vision liquid crystal display comprises: an array substrate 11; a liquid crystal layer 12; a color filter substrate 13; an isolating layer 14; and a grating 15. Compared with a structure of a non-double-vision liquid crystal display, the differences of this structure mainly lie in that the double-vision display effect can be generated through the grating 15 in the case of viewing the double-vision liquid crystal display in a first viewing region 16 and a second viewing region 17, and a first view plane 19 and a second view plane 18 can be seen respectively, as the grating 15 uses opaque material, opaque parts thereof shields a part of an image, thus two different images can be obtained at two different angles.

Touch devices have been widely used in electronic devices. As shown in FIG. 3, a conventional touch device comprises: a first touch electrode 21 comprising a plurality of metal lines parallel to one another and a second touch electrode 22 comprising a plurality of metal lines parallel to one another, wherein each of the metal lines is connected with a touch control module through a leading wire, and the metal lines of the two touch electrodes cross with one other, for example, perpendicular to one another and are located in different layers. Herein, one touch electrode serves as a touch scanning electrode and the other touch electrode serves as a touch sensing electrode, the touch control module drives the metal lines of the touch scanning electrode in a scanning manner and measures whether a capacitance change occurs at a location of each of the metal lines crossing to the driven metal lines of the touch sensing electrode (that is, whether a distance change between the metal lines in the different layers due to touch occurs), through scanning one by one, an accurate touch position can be obtained, and multi-point touch can be achieved.

However, currently it is difficult to touch the double-vision display without adding manufacturing steps and affecting the display effect of the display.

### SUMMARY

Embodiments of the present invention provide a double-vision touch display device and a manufacturing method thereof, which can achieve a touch function without affecting the display effect and adding any manufacturing process.

On the one hand, an embodiment of the present invention provides a double-vision touch display device, comprising: a touch circuit; a grating, wherein, the touch circuit comprises: first touch electrodes, second touch electrodes, and a touch control module, and the first touch electrodes cross with and are insulated from the second touch electrodes, and the first touch electrodes and the second touch electrodes are connected with the touch control module, respectively, and the grating is made from an opaque conductive material and formed with the first touch electrodes synchronously.

On the other hand, an embodiment of the present invention provides a manufacturing method of the above double-vision touch display device, comprising following step: forming the grating and the first touch electrodes through one patterning process.

### BRIEF DESCRIPTION OF THE DRAWINGS

In order to clearly illustrate the technical solution of the embodiments of the invention, the drawings of the embodiments will be briefly described in the following; it is obvious that the described drawings are only related to some embodiments of the invention and thus are not limitative of the invention.

FIG. 1 is a structure view of an existing double-vision liquid crystal display;

FIG. 2 is an arrangement schematic view of a black matrix in an existing liquid crystal display;

FIG. 3 is a schematic view of a first touch electrode and a second touch electrode of an existing touch liquid crystal display;

FIG. 4 is a schematic view of a first touch electrode and a second touch electrode in a double-vision touch display device according to a first embodiment of the present invention;

FIG. 5 is a schematic view showing a structure in which a grating is externally disposed in a double-vision touch display device according to the first embodiment of the present invention;

FIG. 6 is a schematic view showing a structure in which a grating is internally disposed in a double-vision touch display device according to the first embodiment of the present invention;

FIG. 7 is an arrangement schematic view of a black matrix in the double-vision touch display device according to the first embodiment of the present invention;

FIG. 8 is a switch control principle view of a touch signal of another double-vision touch display device according to a second embodiment of the present invention; and

FIG. 9 is a switch control principle view of a touch signal of another double-vision touch display device according to a second embodiment of the present invention.

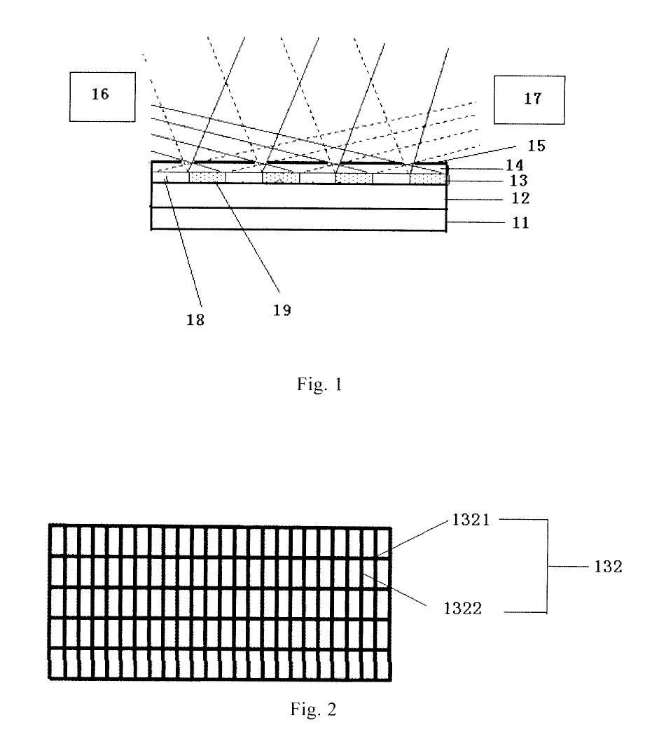

### DETAILED DESCRIPTION

In order to make objects, technical details and advantages of the embodiments of the invention apparent, the technical solutions of the embodiment will be described in a clearly and fully understandable way in connection with the drawings related to the embodiments of the invention. It is obvious that the described embodiments are just a part but not all of the embodiments of the invention. Based on the described embodiments herein, those skilled in the art can obtain other embodiment(s), without any inventive work, which should be within the scope of the invention.

To make one skilled in the art better understand the technical solutions of the present invention, the embodiments of the present invention will be detailed hereinafter in conjunction with the drawings and the specific embodiments.

### A First Embodiment

The first embodiment provides a double-vision touch display device, comprising: a touch circuit; a grating 15, wherein, the touch circuit comprises: a plurality of first touch electrodes 21, a plurality of second touch electrodes 22, and a touch control module; and the first touch electrodes 21 cross with the second touch electrodes 22, and they are connected to the touch control module; the grating 15 is made from opaque conductive material and is formed with the first touch electrodes 21 synchronously (that is, the grating 15 is also used as the first touch electrodes 21).

The working process of the double-vision touch display device will be detailed hereinafter.

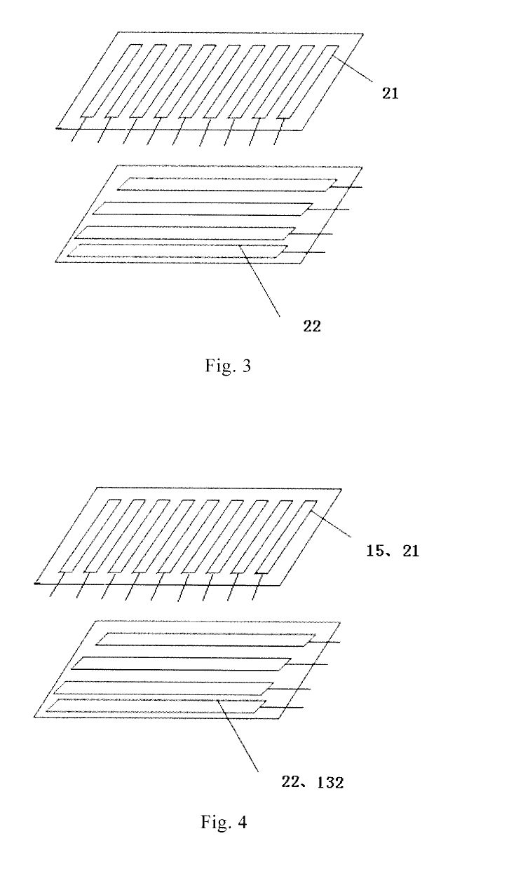

In combination with FIG. 2, through the opaque grating 15, the double-vision touch display device can make a user see two different images at two different angles, that is, a double-vision effect is generated. As the opaque grating 15 has opaque stripes arranged with an interval therebetween, the images seen at two different angles are also different when light passes through the grating 15.

The first touch electrodes 21 cross with the second touch electrodes 22, and they are located in different layers and can form capacitors at crossing positions; wherein, one touch electrode serves as a touch scanning electrode and the other touch electrode serves as a touch sensing electrode, the touch control module drives each of the touch scanning electrodes in a scanning manner, and measures whether a capacitance change occurs at the touch sensing electrode crossing with the driven touch scanning electrode as the touch causes the change of distance between the upper layer electrodes and the lower layer electrodes, and through scanning one by one, an accurate touch position can be obtained, and multi-point touch can be achieved.

In this device, the grating 15 made from conductive material and the first touch electrodes 21 are formed by using one mask synchronously, that is, the grating 15 has actually the functions of the grating and the first touch electrodes at the same time (that is, it is both the grating 15 and the first touch electrodes 21), thus, the structure is simple, the manufacturing process is simplified, the manufacturing cost is saved, and the yield and reliably of the double-vision touch display device can be improved.

Exemplarily, as shown in FIGS. 4 and 5, the double-vision touch display device is a liquid crystal double-vision touch display device, and comprises a color filter substrate 13 which comprises a base 131 and a black matrix 132 disposed on the base, and the grating 15 is disposed at an outer side of the color filter substrate; or the grating 15 is disposed between the base 131 and the black matrix 132 of the color filter substrate 13, and is isolated from the black matrix 132 through a transparent adjustment layer 51. When the grating 15 is located at the outer side (that is, a side away from an array substrate 11 and a liquid crystal layer 12) of the color filter substrate 13, if a distance between the black matrix 132 and the grating 15 is too small, the base 131 of the color filter substrate needs to be thinned, and an isolating layer 14 needs to be disposed; when the grating 15 is disposed between the base 131 and the black matrix 132 , the transparent adjustment layer 51 is disposed between the black matrix 132 and the grating 15, the two situations both aim to keep a certain distance between the black matrix 132 and the grating 15, thus the double-vision displaying can be ensured.

Exemplarily, the grating 15 is made of black metal material, and of course other opaque conductive materials may be also used.

Exemplarily, when the double-vision touch display device is a liquid crystal double-vision touch display device, the second touch electrodes 22 and the black matrix 132 of the color filter substrate 13 are formed synchronously, that is, the black matrix 132 also serves as the second touch electrodes 22, (that is, it is both the black matrix 132 and the second touch electrodes 22), thus the process is further simplified, and the cost can be lowered.

Furthermore, the black matrix 132 is made of black metal material.

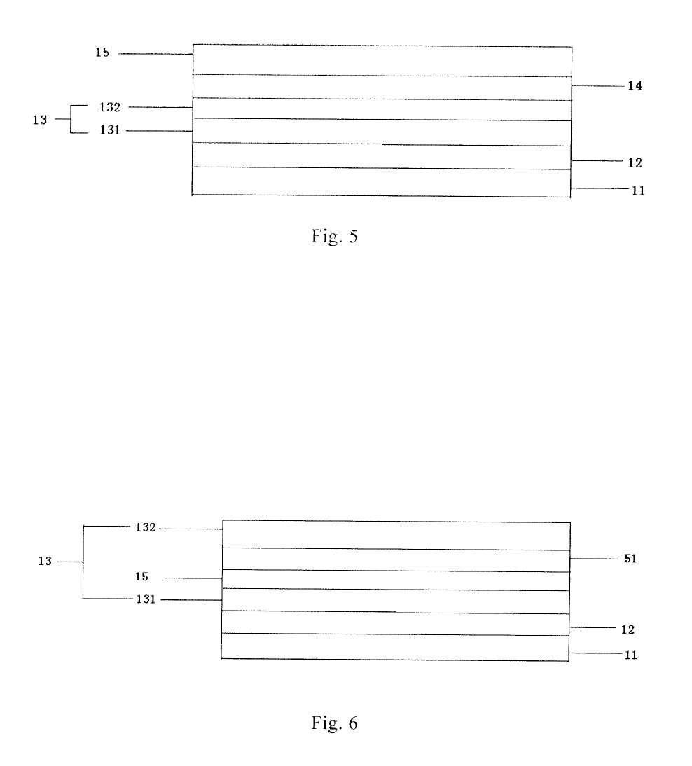

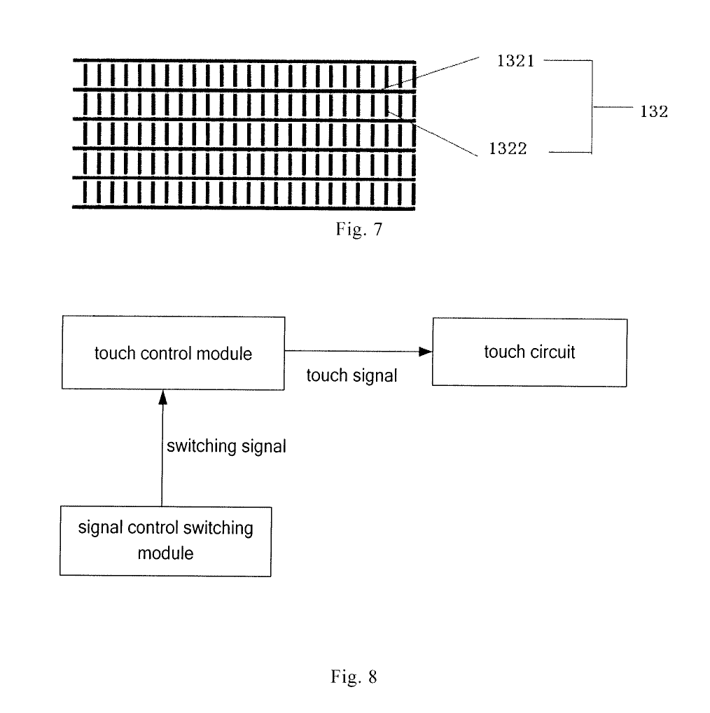

Exemplarily, as shown in FIG. 6, the black matrix 132 comprises a first black matrix 1321 serving as the second touch electrodes 22 and a second black matrix 1322 crossing to the first black matrix and being not connected to the first black matrix. As shown in FIG. 2, in the prior art the black matrix 132 is in a crossing arrangement manner, this will lead to a short circuit due to the crossing of the first black matrix 1321 in the black matrix 132 serving as the second touch electrodes 22 with each of the metal lines. In the arrangement manner in the embodiment of the present invention, the first black matrix 1321 serving as the second touch electrode 22 is made not to connect to the second black matrix 1322, thus the short circuit due to the crossing arrangement of the first black matrix 1321 serving as the second touch electrodes 22 and the metal lines can be avoided.

Exemplarily, the first touch electrodes 21 serve as a touch sensing electrode, and the second touch electrodes 22 serve as a touch scanning electrode; or, the first touch electrodes 21 serve as the touch scanning electrode and the second touch electrodes 22 serve as the touch sensing electrode. The touch scanning electrode and the touch sensing electrode form a capacitive type touch mode.

Exemplarily, the first touch electrodes 21 are arranged perpendicular to the second touch electrodes 22.

### A Second Embodiment

The second embodiment provides a double-vision touch display device, compared with the structure of the display device according to the first embodiment, the differences of the display device according to the second embodiment lie in that it further comprises a signal control switching module connected with a touch control module for achieving a switch between controlling a first view plane 18 and controlling a second view plane 19.

The working process of the double-vision touch display device will be detailed hereinafter.

The embodiment is the same with the first embodiment in the basic principle, while in the embodiment the signal control switching module is added, as shown in FIG. 8, a touch signal only controls one view plane at a certain time, for example, the first view plane 18; at this moment, if the touch signal is intended to control the second view plane 19, the signal control switching module is needed to make the touch signal control the second view plane by using a switching signal, thus, the switch between the touch signal controlling the first view plane 18 and the touch signal controlling the second view plane 19 can be completed. The signal control switching module makes an overall touch system more complete, and avoids the mutual influence between the first view plane and the second view plane during switching a control signal.

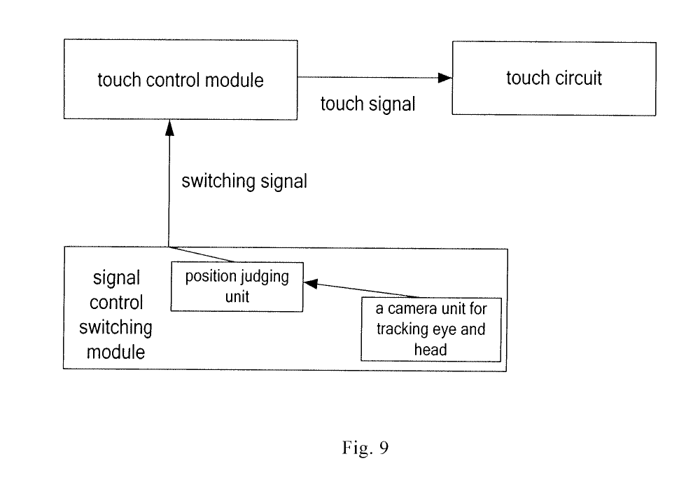

Exemplarily, the signal control switching module comprises a camera unit for tracking eye and head, and a position judging unit or a switch button. As shown in FIG. 9, when the signal control switching module comprises the camera unit for tracking eye and head and the position judging unit, the camera unit is used to track a user's eye and head and the position judging unit is used to judge whether the user is positioned at the first view plane 18 or the second view plane 19, and then the switching signal is output to achieve the switch between the touch signal controlling the first view plane 18 and the touch signal controlling the second view plane 19 according to the user's position. Exemplarily, the switching signal can be a high level or a low level, and is a high level when the user is positioned at the first view plane 18 and is a low level when the user is positioned at the second view plane 19, and when the switching signal is changed from the high level to the low level or from the low level to the high level, the touch signal changes to control the second view plane 19 from the first view plane 18 or to control the first view plane 18 from the second view plane 19, thus, switching a touch of the user between controlling two view planes can be intelligently and automatically performed by this manner.

Exemplarily, in the embodiment of the present invention, the signal control switching module can comprise a switch button, and the switching between the touch signal controlling two view planes can be achieved by a switching operation of a user.

### A Third Embodiment

The third embodiment provides a manufacturing method of a double-vision touch display device according to the first embodiment and the second embodiment, compared with the prior art, in the method of the embodiment, the grating 15 and the first touch electrodes 21 are formed through one patterning process, and the process is simplified and can be achieved easily.

Exemplarily, the double-vision touch display device is a liquid crystal double-vision touch display device comprising a color filter substrate, and the black matrix and the second touch electrodes are formed through one patterning process. Thus, the process can be further simplified and the cost can be saved.

The embodiment of the invention being thus described, it will be obvious that the same may be varied in many ways. Such variations are not to be regarded as a departure from the spirit and scope of the invention, and all such modifications as would be obvious to those skilled in the art are intended to be included within the scope of the following claims.

1. A double-vision touch display device, comprising:
a touch circuit;
a grating,
wherein the touch circuit comprises: first touch electrodes, second touch electrodes and a touch control module, and the first touch electrodes cross with and are insulated from the second touch electrodes, and the first touch electrodes and the second touch electrodes are connected with the touch control module, respectively, and
the grating is made from opaque conductive material and formed with the first touch electrodes synchronously.

2. The double-vision touch display device according to claim 1, wherein the double-vision touch display device displays a first view plane and a second view plane and further comprises a signal control switching module which is connected with the touch control module and is configured to achieve a switch between the touch control module controlling the first view plane and the touch control module controlling the second view plane.

3. The double-vision touch display device according to claim 2, wherein the signal control switching module comprises a camera unit for tracking eye and head, and a position judging unit or a switch button.

4. The double-vision touch display device according to claim 1, wherein the double-vision touch display device is a liquid crystal double-vision touch display device comprising a color filter substrate, and the grating is provided at a light exiting side of the color filter substrate.

5. The double-vision touch display device according to claim 1, wherein the double-vision touch display device is a liquid crystal double-vision touch display device, and the liquid crystal double-vision touch display device comprises a color filter substrate which comprises a base and a black matrix disposed at a light exiting side of the base, and the grating is disposed between the base and the black matrix of the color filter substrate, and a transparent adjustment layer is disposed between the grating and the black matrix and isolates them.

6. The double-vision touch display device according to claim 4, wherein the color filter substrate comprises a base and a black matrix disposed at a light exiting side of the base, and an isolating layer is provided between the grating and the black matrix.

7. The double-vision touch display device according to claim 5, wherein the second touch electrodes and the black matrix of the color filter substrate are formed synchronously, and the black matrix is formed of opaque conductive material.

8. The double-vision touch display device according to claim 7, wherein the black matrix is made of black metal material.

9. The double-vision touch display device according to claim 7, wherein the black matrix comprises a first black matrix serving as the second touch electrodes and a second black matrix crossing with and being not connected to the first black matrix.

10. The double-vision touch display device according to claim 1, wherein the first touch electrodes are arranged perpendicular to the second touch electrodes.

11. A manufacturing method of the double-vision touch display device according to claim 1, comprising the following step:
forming the grating and the first touch electrodes through one patterning process.

12. The manufacturing method of the double-vision touch display device according to claim 11, wherein the double-vision touch display device is a liquid crystal double-vision touch display device comprising a color filter substrate which comprises a base and a black matrix formed on the base, and the manufacturing method further comprises the following step:
forming the black matrix and the second touch electrodes through one patterning process.

13. The double-vision touch display device according to claim 8, wherein the black matrix comprises a first black matrix serving as the second touch electrodes and a second black matrix crossing with and being not connected to the first black matrix.
